(19) United States
(12) Patent Application Publication
(10) Pub. No.: US 2016/0022724 A1
(43) Pub. Date: Jan. 28, 2016

(54) METHODS FOR TREATING PARAMYXOVIRUSES

(71) Applicant: Alios BioPharma, Inc., South San Francisco, CA (US)

(72) Inventors: Sushmita Mukherjee Chanda, Redwood City, CA (US); Qingling Zhang, Burlingame, CA (US); John Fry, San Francisco, CA (US); Lawrence M. Blatt, Healdsburg, CA (US)

(21) Appl. No.: 14/804,034

(22) Filed: Jul. 20, 2015

Related U.S. Application Data
(60) Provisional application No. 62/027,719, filed on Jul. 22, 2014.

(51) Int. Cl. A61K 31/7068; A61K 31/519; A61K 31/4406

(52) U.S. Cl. CPC A61K 31/7068; A61K 31/4406; A61K 31/519

(57) ABSTRACT

Described herein are methods for treating and/or ameliorating a paramyxovirus infection that include using compound (A), or a pharmaceutically acceptable salt thereof.

### INCORPORATION BY REFERENCE TO ANY PRIORITY APPLICATIONS

Any and all applications for which a foreign or domestic priority claim is identified, for example, in the Application Data Sheet or Request as filed with the present application, are hereby incorporated by reference under 37 CFR 1.57, and Rules 4.18 and 20.6.

### FIELD

The present application relates to the fields of chemistry, biochemistry and medicine. More particularly, disclosed herein are methods of ameliorating and/or treating a paramyxovirus infection.

### DESCRIPTION

Respiratory viral infections, including upper and lower respiratory tract viral infections, infects and is the leading cause of death of millions of people each year. Upper respiratory tract viral infections involve the nose, sinuses, pharynx and/or larynx. Lower respiratory tract viral infections involve the respiratory system below the vocal cords, including the trachea, primary bronchi and lungs.

Nucleoside analogs are a class of compounds that have been shown to exert antiviral activity both in vitro and in vivo, and thus, have been the subject of widespread research for the treatment of viral infections. Nucleoside analogs are usually therapeutically inactive compounds that are converted by host or viral enzymes to their respective active anti-metabolites, which, in turn, may inhibit polymerases involved in viral or cell proliferation. The activation occurs by a variety of mechanisms, such as the addition of one or more phosphate groups and, or in combination with, other metabolic processes.

### SUMMARY

Some embodiments described herein generally relate to a method for ameliorating or treating a paramyxovirus infection that can include administering a first dosage of compound (A), or a pharmaceutically acceptable salt thereof, and administering multiple separate second dosages of compound (A), or a pharmaceutically acceptable salt thereof, to a subject suffering from the paramyxovirus infection; and wherein compound (A) is

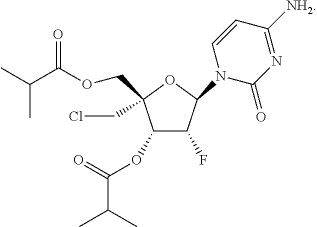 [SMILES: CC(C)C(=O)OC[C@@]1(CCl)O[C@@H](n2ccc(N)nc2=O)[C@H](F)[C@@H]1OC(=O)C(C)C]

Other embodiments described herein generally relate to a method for ameliorating or treating a paramyxovirus infection that can include contacting a cell infected with the paramyxovirus with an effective amount of a compound selected from compound (A) and compound (B), or a pharmaceutically acceptable salt of the foregoing; wherein the method can include administering compound (A), or a pharmaceutically acceptable salt thereof, in a first dosage and administering compound (A), or a pharmaceutically acceptable salt thereof, in multiple separate second dosages; and wherein compound (A) is

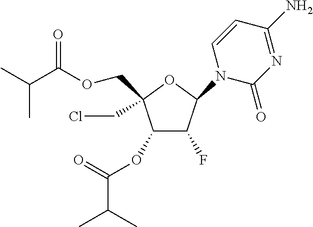 [SMILES: CC(C)C(=O)OC[C@@]1(CCl)O[C@@H](n2ccc(N)nc2=O)[C@H](F)[C@@H]1OC(=O)C(C)C]

and compound (B) is

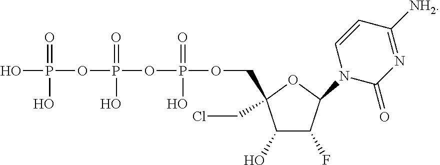 [SMILES: Nc1ccn([C@@H]2O[C@](CCl)(COP(=O)(O)OP(=O)(O)OP(=O)(O)O)[C@@H](O)[C@H]2F)c(=O)n1]

Still other embodiments described herein generally relate to a method for inhibiting the replication of a paramyxovirus that can include contacting a cell infected with the paramyxovirus with an effective amount of a compound selected from compound (A) and compound (B), or a pharmaceutically acceptable salt of the foregoing; wherein the method can include administering compound (A), or a pharmaceutically acceptable salt thereof, in a first dosage and administering compound (A), or a pharmaceutically acceptable salt thereof, in multiple separate second dosages; and wherein compound (A) is

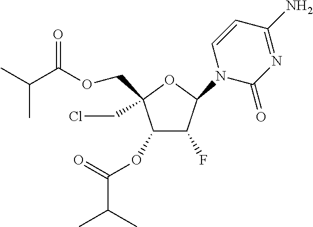 [SMILES: CC(C)C(=O)OC[C@@]1(CCl)O[C@@H](n2ccc(N)nc2=O)[C@H](F)[C@@H]1OC(=O)C(C)C]

and compound (B) is

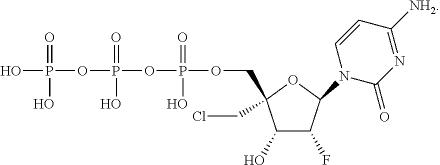 [SMILES: Nc1ccn([C@@H]2O[C@](CCl)(COP(=O)(O)OP(=O)(O)OP(=O)(O)O)[C@@H](O)[C@H]2F)c(=O)n1]

### BRIEF DESCRIPTION OF THE DRAWINGS

FIG. 1 shows the change in RSV viral load following administration of Compound (A) or placebo for 5 Days in ITT-I population.

### DETAILED DESCRIPTION

Paramyxoviridae family is a family of single stranded RNA viruses. Several genera of the paramyxoviridae family include respirovirus, rubulavirus, pneumovirus and metapneumovirus. These viruses can be transmitted person to person via direct or close contact with contaminated respiratory droplets or fomites.

Human Respiratory Syncytial Virus (RSV) is a species of pneumovirus and a negative single-stranded RNA virus. RSV can cause respiratory infections, and can be associated with bronchiolitis and pneumonia. Symptoms of an RSV infection include coughing, sneezing, runny nose, fever, decrease in appetite, sore throat, headache and wheezing. RSV is the most common cause of bronchiolitis and pneumonia in children under one year of age in the world, and can be the cause of tracheobronchitis in older children and adults. In the United States, between 75,000 and 125,000 infants are hospitalized each year with RSV. Among adults older than 65 years of age, an estimated 14,000 deaths and 177,000 hospitalizations have been attributed to RSV.

Treatment options for people infected with RSV are currently limited. Antibiotics, usually prescribed to treat bacterial infections, and over-the-counter medication are not effective in treating RSV and may help only to relieve some of the symptoms. In severe cases, a nebulized bronchodilator, such as albuterol, may be prescribed to relieve some of the symptoms, such as wheezing. RespiGam® (RSV-IGIV, Medlmmune, approved for high risk children younger than 24 months of age) and Synagis® (palivizumab, Medlmmune, approved for high risk children younger than 24 months of age) have been approved for prophylactic use against RSV, and Virzole® (ribavirin by aerosol, ICN pharmaceuticals) have been approved for the treatment of RSV.

Parainfluenza viruses are typically negative-sense RNA viruses. Species of respirovirus include human parainfluenza viruses 1 and 3; and species of rubulavirus include human parainfluenza viruses 2 and 4. Human parainfluenza virus includes four serotypes types (HPIV-1, HPIV-2, HPIV-3 and HPIV-4), and human parainfluenza virus 4 (HPIV-4) include two antigenic subgroups, A and B. Human parainfluenza viruses can cause upper and lower respiratory tract infections. Human parainfluenza virus 1 (HPIV-1) and human parainfluenza virus 2 (HPIV-2) can be associated with croup; human parainfluenza virus 3 (HPIV-3) can be associated with bronchiolitis and pneumonia. According to the Centers of Disease Control and Prevention (CDC), there are no vaccines against human parainfluenza viruses.

A species of metapneumovirus is human metapneumovirus. Human metapneumovirus is a negative single-stranded RNA virus. Human metapneumovirus can cause respiratory tract infections, such as upper and lower respiratory tract infections in human, for example young children.

Respiratory infections include colds, croup, pneumonia, bronchitis, tracheobronchitis and bronchiolitis. Symptoms can include a cough, runny nose, nasal congestion, sore throat, fever, difficulty breathing, abnormally rapid breathing, wheezing vomiting, diarrhea and ear infections.

Some embodiments described herein relate to a method for ameliorating or treating a paramyxovirus infection that can include administering a first dosage of compound (A), or a pharmaceutically acceptable salt thereof, and administering multiple separate second dosages of compound (A), or a pharmaceutically acceptable salt thereof, to a subject suffering from the paramyxovirus infection. Other embodiments described herein relate to using a first dosage compound (A), or a pharmaceutically acceptable salt thereof, and multiple separate second dosages of compound (A), or a pharmaceutically acceptable salt thereof, in the manufacture of a medicament for ameliorating and/or treating a paramyxovirus infection in a subject suffering from the paramyxovirus infection. Still other embodiments described herein relate to a first dosage of compound (A), or a pharmaceutically acceptable salt thereof, and multiple separate second dosages of compound (A), or a pharmaceutically acceptable salt thereof, that can be used for ameliorating and/or treating a paramyxovirus infection in a subject suffering from the paramyxovirus infection.

Some embodiments disclosed herein relate to a method of ameliorating and/or treating a paramyxovirus infection that can include contacting a cell infected with the paramyxovirus with an effective amount of a compound selected from compound (A) and compound (B), or a pharmaceutically acceptable salt of the foregoing; wherein the method can include administering compound (A), or a pharmaceutically acceptable salt thereof, in a first dosage and administering compound (A), or a pharmaceutically acceptable salt thereof, in multiple separate second dosages. Other embodiments described herein relate to using a compound selected from compound (A) and compound (B), or a pharmaceutically acceptable salt of the foregoing, in the manufacture of a medicament for ameliorating and/or treating a paramyxovirus infection that can include contacting a cell infected with the paramyxovirus with an effective amount of said compound and/or compounds; and wherein the use can include administering compound (A), or a pharmaceutically acceptable salt thereof, in a first dosage and administering compound (A), or a pharmaceutically acceptable salt thereof, in multiple separate second dosages. Still other embodiments described herein relate to a compound selected from compound (A) and compound (B), or a pharmaceutically acceptable salt of the foregoing, that can be used for ameliorating and/or treating a paramyxovirus infection by contacting a cell infected with the paramyxovirus with an effective amount of said compound and/or compounds; and wherein the use can include administering compound (A), or a pharmaceutically acceptable salt thereof, in a first dosage and administering compound (A), or a pharmaceutically acceptable salt thereof, in multiple separate second dosages.

Some embodiments disclosed herein relate to a method of inhibiting replication of a paramyxovirus that can include contacting a cell infected with the paramyxovirus with an effective amount of compound (A) and/or compound (B), or a pharmaceutically acceptable salt of the foregoing; and wherein the method can include administering compound (A), or a pharmaceutically acceptable salt thereof, in a first dosage and administering compound (A), or a pharmaceutically acceptable salt thereof, in multiple separate second dosages. Other embodiments described herein relate to using compound (A) and/or compound (B), or a pharmaceutically acceptable salt of the foregoing, in the manufacture of a medicament for inhibiting replication of a paramyxovirus that can include contacting a cell infected with the paramyxovirus with an effective amount of said compound and/or compounds; and wherein the use can include administering compound (A), or a pharmaceutically acceptable salt thereof, in a first dosage and administering compound (A), or a pharmaceutically acceptable salt thereof, in multiple separate second dosages. Still other embodiments described herein relate to compound (A) and/or compound (B), or a pharmaceutically acceptable salt of the foregoing, that can be used for inhibiting replication of a paramyxovirus by contacting a cell infected with the paramyxovirus with an effective amount of compound and/or compounds; and wherein the use can include administering compound (A), or a pharmaceutically acceptable salt thereof, in a first dosage and administering compound (A), or a pharmaceutically acceptable salt thereof, in multiple separate second dosages.

Some embodiments described herein relate to a method of inhibiting a paramyxovirus polymerase can include contacting a cell infected with a paramyxovirus with an effective amount of compound (A) and/or compound (B), or a pharmaceutically acceptable salt of the foregoing; and wherein the method can include administering compound (A), or a pharmaceutically acceptable salt thereof, in a first dosage and administering compound (A), or a pharmaceutically acceptable salt thereof, in multiple separate second dosages. Other embodiments described herein relate to using compound (A) and/or compound (B), or a pharmaceutically acceptable salt of the foregoing, in the manufacture of a medicament for inhibiting a paramyxovirus polymerase that can include contacting a cell infected with the paramyxovirus with an effective amount of said compound and/or compounds; and wherein the use can include administering compound (A), or a pharmaceutically acceptable salt thereof, in a first dosage and administering compound (A), or a pharmaceutically acceptable salt thereof, in multiple separate second dosages. Still other embodiments described herein relate to compound (A) and/or compound (B), or a pharmaceutically acceptable salt of the foregoing, that can be used for inhibiting a paramyxovirus polymerase that can include contacting a cell infected with the paramyxovirus with an effective amount of said compound and/or compounds; and wherein the use can include administering compound (A), or a pharmaceutically acceptable salt thereof, in a first dosage and administering compound (A), or a pharmaceutically acceptable salt thereof, in multiple separate second dosages.

Some embodiments described herein relate to a method of ameliorating and/or treating a respiratory infection (for example, an upper and/or lower respiratory infection) in a subject suffering from the respiratory infection, wherein the respiratory infection is caused by a paramyxovirus infection, that can include administering a first dosage of compound (A), or a pharmaceutically acceptable salt thereof, and administering multiple separate second dosages of compound (A), or a pharmaceutically acceptable salt thereof. Other embodiments described herein relate to a method of ameliorating and/or treating a respiratory infection in a subject suffering from the respiratory infection, wherein the respiratory infection is caused by a paramyxovirus infection, that can include contacting a cell infected with paramyxovirus in the subject with an effective amount of compound (A) and/or compound (B), or a pharmaceutically acceptable salt of the foregoing; and wherein the method can include administering compound (A), or a pharmaceutically acceptable salt thereof, in a first dosage and administering compound (A), or a pharmaceutically acceptable salt thereof, in multiple separate second dosages. Still other embodiments described herein relate to using compound (A) and/or compound (B), or a pharmaceutically acceptable salt of the foregoing, in the manufacture of a medicament for ameliorating and/or treating a respiratory infection, wherein the respiratory infection is due to a paramyxovirus infection, that can include administering a first dosage of compound (A), or a pharmaceutically acceptable salt thereof, and administering multiple separate second dosages of compound (A), or a pharmaceutically acceptable salt thereof. Yet still other embodiments described herein relate to compound (A) and/or compound (B), or a pharmaceutically acceptable salt of the foregoing, that can be used for ameliorating and/or treating a respiratory infection in a subject suffering from the respiratory infection, wherein the respiratory infection is from a paramyxovirus infection, that can include contacting a cell infected with the paramyxovirus in the subject with an effective amount of compound (A) and/or compound (B), or a pharmaceutically acceptable salt of the foregoing; and wherein the use can include administering compound (A), or a pharmaceutically acceptable salt thereof, in a first dosage and administering compound (A), or a pharmaceutically acceptable salt thereof, in multiple separate second dosages. Some embodiments described herein relate to compound (A) and/or compound (B), or a pharmaceutically acceptable salt of the foregoing, that can be used for ameliorating and/or treating a respiratory infection in a subject suffering from the respiratory infection, wherein the respiratory infection is from a paramyxovirus infection, that can include administering a first dosage of compound (A), or a pharmaceutically acceptable salt thereof, and administering multiple separate second dosages of compound (A), or a pharmaceutically acceptable salt thereof. Other embodiments described herein relate to compound (A) and/or compound (B), or a pharmaceutically acceptable salt of the foregoing, that can be used for ameliorating and/or treating a respiratory infection in a subject suffering from the respiratory infection, wherein the respiratory infection is from a paramyxovirus infection, that can include contacting a cell infected with the paramyxovirus in the subject with an effective amount of compound (A) and/or compound (B), or a pharmaceutically acceptable salt of the foregoing; and wherein the use can include administering compound (A), or a pharmaceutically acceptable salt thereof, in a first dosage and administering compound (A), or a pharmaceutically acceptable salt thereof, in multiple separate second dosages. Examples of respiratory infections include those described herein, such as, colds, croup, pneumonia, bronchitis, tracheobronchitis and bronchiolitis. A non-limiting list of symptoms of a respiratory infection can include a cough, runny nose, nasal congestion, sore throat, fever, difficulty breathing, abnormally rapid breathing, wheezing vomiting, diarrhea and ear infections.

In some embodiments, a method and/or use described herein can be used to ameliorate and/or treat a RSV infection, a respiratory infection attributable to a RSV infection and/or one or more symptoms of a RSV infection. A compound described herein may be active against more than one type of RSV. In some embodiments, a method and/or use described herein can be used to ameliorate and/or treat an infection caused by RSV strain A. In other embodiments, a method and/or use described herein can be used to ameliorate and/or treat an infection caused by RSV strain B. In still other embodiments, a method and/or use described herein can be used to ameliorate and/or treat an infection caused by RSV strains A and B. In some embodiments, a method and/or use described herein can be used to ameliorate and/or treat a metapneumovirus infection (for example, a human metapneumovirus infection), a respiratory infection attributable to a metapneumovirus infection and/or one or more symptoms of a metapneumovirus infection. In some embodiments, a method and/or use described herein can be used to ameliorate and/or treat a human parainfluenza virus infection (for example, a HPIV-1, HPIV-2, HPIV-3 and HPIV-4 infection), a respiratory infection attributable to a human parainfluenza infection and/or one or more symptoms of a human parainfluenza infection.

Some embodiments described herein relate to a method for preventing a paramyxovirus infection. In some embodiment, a first dosage of compound (A), or a pharmaceutically acceptable salt thereof, and multiple separate second dosages of compound (A), or a pharmaceutically acceptable salt thereof, can be administered to a subject to prevent a paramyxovirus infection (for example, as prophylactic treatment). In other embodiments a first dosage compound (A), or a pharmaceutically acceptable salt thereof, and multiple separate second dosages of compound (A), or a pharmaceutically acceptable salt thereof, can be manufactured into a medicament for preventing a paramyxovirus infection in a subject. In still other embodiments a first dosage compound (A), or a pharmaceutically acceptable salt thereof, and multiple separate second dosages of compound (A), or a pharmaceutically acceptable salt thereof, can be used for preventing a paramyxovirus infection.

The compounds (A) and (B), or a pharmaceutically acceptable salt of the foregoing, are described in U.S. Publication Nos. 2013/0165400 and 2015/0051167 and International Publication Nos. WO 2013/142525 and WO 2013/142525, all of which are hereby incorporated by reference in their entireties. Those skilled in the art understand that once compound (A), or a pharmaceutically acceptable salt thereof, is absorbed, the groups attached to 3′ and 5′ positions can be easily removed by esterases, proteases and/or other enzymes. Once inside the cell, the triphosphate (compound (B), or a pharmaceutically acceptable salt thereof) can be formed via metabolization by cellular enzymes. Compound (B), or a pharmaceutically acceptable salt thereof, inhibits RNA polymerase activity via a chain termination mechanism, and has a half life of approximately 17.6 hours.

In some embodiments, the first dosage of compound (A), or a pharmaceutically salt thereof, can include an amount of compound (A), or a pharmaceutically salt thereof, in the range of 1000 mg to 5 mg. In other embodiments, the first dosage of compound (A), or a pharmaceutically salt thereof, can include an amount of compound (A), or a pharmaceutically salt thereof, in the range of 800 mg to 700 mg. In still other embodiments, the first dosage of compound (A), or a pharmaceutically salt thereof, can include an amount of compound (A), or a pharmaceutically salt thereof, in the range of 725 mg to 775 mg. In yet still other embodiments, the first dosage of compound (A), or a pharmaceutically salt thereof, can include an amount of compound (A), or a pharmaceutically salt thereof, in the range of 325 mg to 425 mg. In some embodiments, the first dosage of compound (A), or a pharmaceutically salt thereof, can include an amount of compound (A), or a pharmaceutically salt thereof, in the range of 350 mg to 400 mg. In other embodiments, the first dosage of compound (A), or a pharmaceutically salt thereof, can include an amount of compound (A), or a pharmaceutically salt thereof, in the range of 100 mg to 200 mg. In still other embodiments, the first dosage of compound (A), or a pharmaceutically salt thereof, can include an amount of compound (A), or a pharmaceutically salt thereof, in the range of 125 mg to 175 mg. In yet still other embodiments, the first dosage of compound (A), or a pharmaceutically salt thereof, can include an amount of compound (A), or a pharmaceutically salt thereof, in the range of 450 mg to 550 mg. In some embodiments, the first dosage of compound (A), or a pharmaceutically salt thereof, can include an amount of compound (A), or a pharmaceutically salt thereof, in the range of 475 mg to 525 mg. In other embodiments, the first dosage of compound (A), or a pharmaceutically salt thereof, can include an amount of compound (A), or a pharmaceutically salt thereof, in the range of 5 mg to 175 mg. In still other embodiments, the first dosage of compound (A), or a pharmaceutically salt thereof, can include an amount of compound (A), or a pharmaceutically salt thereof, in the range of 15 mg to 150 mg. In yet still other embodiments, the first dosage of compound (A), or a pharmaceutically salt thereof, can include an amount of compound (A), or a pharmaceutically salt thereof, in the range of 20 mg to 130 mg. In some embodiments, the first dosage of compound (A), or a pharmaceutically salt thereof, can include an amount of compound (A), or a pharmaceutically salt thereof, in the range of 700 mg to 1600 mg.

In some embodiments, each second dosage of compound (A), or a pharmaceutically salt thereof, can include an amount of compound (A), or a pharmaceutically salt thereof, in the range of 1000 mg to 5 mg. In other embodiments, each second dosage of compound (A), or a pharmaceutically salt thereof, can include an amount of compound (A), or a pharmaceutically salt thereof, in the range of 800 mg to 700 mg. In still other embodiments, each second dosage of compound (A), or a pharmaceutically salt thereof, can include an amount of compound (A), or a pharmaceutically salt thereof, in the range of 725 mg to 775 mg. In yet still other embodiments, each second dosage of compound (A), or a pharmaceutically salt thereof, can include an amount of compound (A), or a pharmaceutically salt thereof, in the range of 325 mg to 425 mg. In some embodiments, each second dosage of compound (A), or a pharmaceutically salt thereof, can include an amount of compound (A), or a pharmaceutically salt thereof, in the range of 350 mg to 400 mg. In other embodiments, each second dosage of compound (A), or a pharmaceutically salt thereof, can include an amount of compound (A), or a pharmaceutically salt thereof, in the range of 100 mg to 200 mg. In still other embodiments, each second dosage of compound (A), or a pharmaceutically salt thereof, can include an amount of compound (A), or a pharmaceutically salt thereof, in the range of 125 mg to 175 mg. In yet still other embodiments, each second dosage of compound (A), or a pharmaceutically salt thereof, can include an amount of compound (A), or a pharmaceutically salt thereof, in the range of 450 mg to 550 mg. In some embodiments, each second dosage of compound (A), or a pharmaceutically salt thereof, can include an amount of compound (A), or a pharmaceutically salt thereof, in the range of 475 mg to 525 mg. In other embodiments, each second dosage of compound (A), or a pharmaceutically salt thereof, can include an amount of compound (A), or a pharmaceutically salt thereof, in the range of 5 mg to 175 mg. In still other embodiments, each second dosage of compound (A), or a pharmaceutically salt thereof, can include an amount of compound (A), or a pharmaceutically salt thereof, in the range of 15 mg to 150 mg. In yet still other embodiments, each second dosage of compound (A), or a pharmaceutically salt thereof, can include an amount of compound (A), or a pharmaceutically salt thereof, in the range of 20 mg to 130 mg. In some embodiments, each second dosage of compound (A), or a pharmaceutically salt thereof, can include an amount of compound (A), or a pharmaceutically salt thereof, in the range of 700 mg to 1600 mg.

In some embodiments, the first dosage of compound (A), or a pharmaceutically salt thereof, can include an amount of compound (A), or a pharmaceutically salt thereof, of 375±10 mg. In other embodiments the first dosage of compound (A), or a pharmaceutically salt thereof, can include an amount of compound (A), or a pharmaceutically salt thereof, of 750±10 mg. In still other embodiments, the first dosage of compound (A), or a pharmaceutically salt thereof, can include an amount of compound (A), or a pharmaceutically salt thereof, of 150±10 mg. In some embodiments, the first dosage of compound (A), or a pharmaceutically salt thereof, can include an amount of compound (A), or a pharmaceutically salt thereof, of 500±10 mg. In other embodiments, the first dosage of compound (A), or a pharmaceutically salt thereof, can include an amount of compound (A), or a pharmaceutically salt thereof, of at least 25±2 mg. In still other embodiments, the first dosage of compound (A), or a pharmaceutically salt thereof, can include an amount of compound (A), or a pharmaceutically salt thereof, of at least 50±2 mg. In yet still other embodiments, the first dosage of compound (A), or a pharmaceutically salt thereof, can include an amount of compound (A), or a pharmaceutically salt thereof, of at least 2±0.5 mg. In some embodiments, the first dosage of compound (A), or a pharmaceutically salt thereof, can include an amount of compound (A), or a pharmaceutically salt thereof, of at least 5±0.5 mg. In other embodiments, the first dosage of compound (A), or a pharmaceutically salt thereof, can include an amount of compound (A), or a pharmaceutically salt thereof, of at least 6±0.5 mg. In still other embodiments, the first dosage of compound (A), or a pharmaceutically salt thereof, can include an amount of compound (A), or a pharmaceutically salt thereof, of at least 10±0.5 mg. In yet still other embodiments, the first dosage of compound (A), or a pharmaceutically salt thereof, can include an amount of compound (A), or a pharmaceutically salt thereof, of at least 25±1.0 mg. In some embodiments, the first dosage of compound (A), or a pharmaceutically salt thereof, can include an amount of compound (A), or a pharmaceutically salt thereof, of at least 100±2 mg.

In some embodiments, each second dosage of compound (A), or a pharmaceutically salt thereof, can include an amount of compound (A), or a pharmaceutically salt thereof, of 375±10 mg. In other embodiments each second dosage of compound (A), or a pharmaceutically salt thereof, can include an amount of compound (A), or a pharmaceutically salt thereof, of 750±10 mg. In still other embodiments, each second dosage of compound (A), or a pharmaceutically salt thereof, can include an amount of compound (A), or a pharmaceutically salt thereof, of 150±10 mg. In some embodiments, each second dosage of compound (A), or a pharmaceutically salt thereof, can include an amount of compound (A), or a pharmaceutically salt thereof, of 500±10 mg. In some embodiments, each second dosage of compound (A), or a pharmaceutically salt thereof, can include an amount of compound (A), or a pharmaceutically salt thereof, of at least 25±2 mg. In other embodiments, each second dosage of compound (A), or a pharmaceutically salt thereof, can include an amount of compound (A), or a pharmaceutically salt thereof, of at least 50±2 mg. In still other embodiments, each second dosage of compound (A), or a pharmaceutically salt thereof, can include an amount of compound (A), or a pharmaceutically salt thereof, of at least 2±0.5 mg. In yet still other embodiments, each second dosage of compound (A), or a pharmaceutically salt thereof, can include an amount of compound (A), or a pharmaceutically salt thereof, of at least 5±0.5 mg. In some embodiments, each second dosage of compound (A), or a pharmaceutically salt thereof, can include an amount of compound (A), or a pharmaceutically salt thereof, of at least 6±0.5 mg. In other embodiments, each second dosage of compound (A), or a pharmaceutically salt thereof, can include an amount of compound (A), or a pharmaceutically salt thereof, of at least 10±0.5 mg. In still other embodiments, each second dosage of compound (A), or a pharmaceutically salt thereof, can include an amount of compound (A), or a pharmaceutically salt thereof, of at least 25±1.0 mg. In yet still other embodiments, each second dosage of compound (A), or a pharmaceutically salt thereof, can include an amount of compound (A), or a pharmaceutically salt thereof, of at least 100±2 mg.

In some embodiments, the first dosage of compound (A), or a pharmaceutically salt thereof, can include an amount of compound (A), or a pharmaceutically salt thereof, in the range of 8 mg/kg to 15 mg/kg. In other embodiments, the first dosage of compound (A), or a pharmaceutically salt thereof, can include an amount of compound (A), or a pharmaceutically salt thereof, in the range of 9 mg/kg to 13 mg/kg. In still other embodiments, the first dosage of compound (A), or a pharmaceutically salt thereof, can include an amount of compound (A), or a pharmaceutically salt thereof, in the range of 1 mg/kg to 20 mg/kg. In yet still other embodiments, the first dosage of compound (A), or a pharmaceutically salt thereof, can include an amount of compound (A), or a pharmaceutically salt thereof, in the range of 5 mg/kg to 18 mg/kg. In some embodiments, the first dosage of compound (A), or a pharmaceutically salt thereof, can include an amount of compound (A), or a pharmaceutically salt thereof, in the range of 5 mg/kg to 30 mg/kg. In other embodiments, the first dosage of compound (A), or a pharmaceutically salt thereof, can include an amount of compound (A), or a pharmaceutically salt thereof, in the range of 2 mg/kg to 50 mg/kg. In still other embodiments, the first dosage of compound (A), or a pharmaceutically salt thereof, can include an amount of compound (A), or a pharmaceutically salt thereof, in the range of 10 mg/kg to 25 mg/kg. In still other embodiments, the first dosage of compound (A), or a pharmaceutically salt thereof, can include an amount of compound (A), or a pharmaceutically salt thereof, in the range of 5 mg/kg to 75 mg/kg. In yet still other embodiments, the first dosage of compound (A), or a pharmaceutically salt thereof, can include an amount of compound (A), or a pharmaceutically salt thereof, in the range of 1 mg/kg to 50 mg/kg.

In some embodiments, each second dosage of compound (A), or a pharmaceutically salt thereof, can include an amount of compound (A), or a pharmaceutically salt thereof, in the range of 8 mg/kg to 15 mg/kg. In other embodiments, each second dosage of compound (A), or a pharmaceutically salt thereof, can include an amount of compound (A), or a pharmaceutically salt thereof, in the range of 9 mg/kg to 13 mg/kg. In still other embodiments, each second dosage of compound (A), or a pharmaceutically salt thereof, can include an amount of compound (A), or a pharmaceutically salt thereof, in the range of 1 mg/kg to 20 mg/kg. In yet still other embodiments, each second dosage of compound (A), or a pharmaceutically salt thereof, can include an amount of compound (A), or a pharmaceutically salt thereof, in the range of 5 mg/kg to 18 mg/kg. In some embodiments, each second dosage of compound (A), or a pharmaceutically salt thereof, can include an amount of compound (A), or a pharmaceutically salt thereof, in the range of 5 mg/kg to 30 mg/kg. In other embodiments, each second dosage of compound (A), or a pharmaceutically salt thereof, can include an amount of compound (A), or a pharmaceutically salt thereof, in the range of 2 mg/kg to 50 mg/kg. In still other embodiments, each second dosage of compound (A), or a pharmaceutically salt thereof, can include an amount of compound (A), or a pharmaceutically salt thereof, in the range of 10 mg/kg to 25 mg/kg. In yet still other embodiments, each second dosage of compound (A), or a pharmaceutically salt thereof, can include an amount of compound (A), or a pharmaceutically salt thereof, in the range of 5 mg/kg to 75 mg/kg. In some embodiments, each second dosage of compound (A), or a pharmaceutically salt thereof, can include an amount of compound (A), or a pharmaceutically salt thereof, in the range of 1 mg/kg to 50 mg/kg.

In some embodiments, the first dosage of compound (A), or a pharmaceutically salt thereof, can include an amount of compound (A), or a pharmaceutically salt thereof, of 2±0.5 mg/kg. In other embodiments, the first dosage of compound (A), or a pharmaceutically salt thereof, can include an amount of compound (A), or a pharmaceutically salt thereof, of 5±0.5 mg/kg. In still other embodiments, the first dosage of compound (A), or a pharmaceutically salt thereof, can include an amount of compound (A), or a pharmaceutically salt thereof, of 10±0.5 mg/kg. In yet still other embodiments, the first dosage of compound (A), or a pharmaceutically salt thereof, can include an amount of compound (A), or a pharmaceutically salt thereof, of 25±1.0 mg/kg. In some embodiments, the first dosage of compound (A), or a pharmaceutically salt thereof, can include an amount of compound (A), or a pharmaceutically salt thereof, of 30±1.0 mg/kg. In other embodiments, the first dosage of compound (A), or a pharmaceutically salt thereof, can include an amount of compound (A), or a pharmaceutically salt thereof, of 50±2.0 mg/kg. In still other embodiments, the first dosage of compound (A), or a pharmaceutically salt thereof, can include an amount of compound (A), or a pharmaceutically salt thereof, more than 50 mg/kg.

In some embodiments, each second dosage of compound (A), or a pharmaceutically salt thereof, can include an amount of compound (A), or a pharmaceutically salt thereof, of 2±0.5 mg/kg. In other embodiments, each second dosage of compound (A), or a pharmaceutically salt thereof, can include an amount of compound (A), or a pharmaceutically salt thereof, of 5±0.5 mg/kg. In still other embodiments, each second dosage of compound (A), or a pharmaceutically salt thereof, can include an amount of compound (A), or a pharmaceutically salt thereof, of 10±0.5 mg/kg. In yet still other embodiments, each second dosage of compound (A), or a pharmaceutically salt thereof, can include an amount of compound (A), or a pharmaceutically salt thereof, of 25±1.0 mg/kg. In some embodiments, each second dosage of compound (A), or a pharmaceutically salt thereof, can include an amount of compound (A), or a pharmaceutically salt thereof, of 30±1.0 mg/kg. In other embodiments, each second dosage of compound (A), or a pharmaceutically salt thereof, can include an amount of compound (A), or a pharmaceutically salt thereof, of 50±2.0 mg/kg. In still other embodiments, each second dosage of compound (A), or a pharmaceutically salt thereof, can include an amount of compound (A), or a pharmaceutically salt thereof, more than 50 mg/kg.

In some embodiments, different amounts of compound (A), or a pharmaceutically acceptable salt thereof, can be given during treatment. In other embodiments, the same amounts of compound (A), or a pharmaceutically acceptable salt thereof, can be given during treatment. In some embodiments, one or more “loading” dosages that can include an amount(s) of compound (A), or a pharmaceutically acceptable salt thereof, can be given followed by several “maintenance” dosages that can include an amount(s) of compound (A), or a pharmaceutically acceptable salt thereof. The terms “loading dosage” and “maintenance dosage” are used herein as understood by those skilled in the art. A “loading dosage” is an amount of a compound provided for the purpose of establishing a therapeutic level of the compound in the target tissue (for example, the lung). A “maintenance dosage” is an amount of a compound provided to maintain a desired level of the compound in the target tissue (such as the lung). In some embodiments, the amount of the loading dosage can be greater than the amount of each maintenance dosage. In other embodiments, the amount of the loading dosage can be the same as the amount of each maintenance dosage. In some embodiments, the amount of compound being maintained is the active metabolite in the target tissue (for example, an amount of compound (B), or a pharmaceutically acceptable salt thereof, in lung tissue). Those skilled in the art understand that the loading dosage that may include a single dosage or multiple dosages is given for a first period of time followed by one or more maintenance dosages for a second period of time. The loading and maintenance dosages can be adjusted so that the peak plasma concentrations (Cmax) and/or the plasma area under the curve (AUC) are the same following every dose at a certain time period.

In some embodiments, a first dosage of compound (A), or a pharmaceutically acceptable salt thereof, can be divided between multiple dosages. For example, a first dosage of compound (A), or a pharmaceutically acceptable salt thereof, can be divided between two dosages, wherein each dosage can include an amount of compound (A), or a pharmaceutically acceptable salt therein, in the range of 325 mg to 425 mg (such as 375±10 mg). In other embodiments, a first dosage of compound (A), or a pharmaceutically acceptable salt thereof, can be a single dosage that can include an amount of compound (A), or a pharmaceutically acceptable salt therein, in the range of 700 mg to 800 mg (for example, 750±10 mg). When the first dosage of compound (A), or a pharmaceutically acceptable salt thereof, is divided between multiple dosages, the amount of compound (A), or a pharmaceutically acceptable salt thereof, in each dosage may be the same. Alternatively, the amount of compound (A), or a pharmaceutically acceptable salt thereof, in each dosage may differ from one or more of the other dosages. In some embodiments, the first dosage can be a loading dose.

After the first dosage of compound (A), or a pharmaceutically acceptable salt thereof, several second dosages of compound (A), or a pharmaceutically acceptable salt thereof, can be provided. In some embodiments, each second dosage can include an amount of compound (A), or a pharmaceutically acceptable salt thereof, in the range of 100 mg to 200 mg (such as 150±10 mg). In other embodiments, each second dosage can include an amount of compound (A), or a pharmaceutically acceptable salt thereof, in the range of 450 mg to 550 mg (for example, 500±10 mg). In still other embodiments, each second dosage can include an amount of compound (A), or a pharmaceutically acceptable salt thereof, in the range of 325 mg to 425 mg (such as 375±10 mg). In yet still other embodiments, each second dosage can include an amount of compound (A), or a pharmaceutically acceptable salt thereof, in the range of 100 mg to 25 mg (such as 50±5 mg). Each second dosage of compound (A), or a pharmaceutically acceptable salt thereof, can include the same amount of compound (A), or a pharmaceutically acceptable salt thereof, or a different amount of compound (A), or a pharmaceutically acceptable salt thereof, from another second dosage of compound (A), or a pharmaceutically acceptable salt thereof. In some embodiments, the second dosage(s) can be maintenance dosage(s).

As described herein, multiple second dosages of compound (A), or a pharmaceutically acceptable salt thereof, can be provided. In some embodiments, the number of second dosages can be in the range of 2 to 20 separate second dosages of compound (A), or a pharmaceutically acceptable salt thereof. In other embodiments, the number of second dosages can be in the range of 2 to 15 separate second dosages of compound (A), or a pharmaceutically acceptable salt thereof. In still other embodiments, the number of second dosages can be in the range of 2 to 12 separate second dosages of compound (A), or a pharmaceutically acceptable salt thereof. In still other embodiments, the number of second dosages can be in the range of 2 to 10 separate second dosages of compound (A), or a pharmaceutically acceptable salt thereof. In some embodiments, the number of second dosages can be more than 2 separate second dosages of compound (A), or a pharmaceutically acceptable salt thereof. In other embodiments, the number of second dosages can be more than 5 separate second dosages of compound (A), or a pharmaceutically acceptable salt thereof. In still other embodiments, the number of second dosages can be more than 8 separate second dosages of compound (A), or a pharmaceutically acceptable salt thereof.

The frequency and length of administration of compound (A), or a pharmaceutically salt thereof, can vary. In some embodiments, compound (A), or a pharmaceutically salt thereof, can be dosed once daily. In other embodiments, compound (A), or a pharmaceutically salt thereof, can be dosed twice daily. For example, compound (A), or a pharmaceutically salt thereof, can be provided at a first time period and then at a second time period, wherein the first time period and the second time period are separated by at least 8 hours. In some embodiments, the first dosage of compound (A), or a pharmaceutically acceptable salt thereof, can be given in a single dosage once daily. In other embodiments, the first dosage of compound (A), or a pharmaceutically acceptable salt thereof, can be given in two dosages at different times. As an example, one of the first dosages can be given at a first time period and other of the first dosages can be given at a second time period, wherein the two time periods are separated by one or more hours (for example, separated by 8-14 hours). In some embodiments, the two dosages of the first dosage are separated by approximately 12 hours.

The initial second dosage and subsequent second dosages can be administered at various times. In some embodiments, the initial second dosage can be provided in the range of 8 hours to 14 hours after completion of the first dosage (such as after the final dosage of the first dosage). In some embodiments, the initial second dosage can be provided approximately 12 hours after completion of the first dosage. The subsequent second dosages can be provided at approximate regular intervals following the initial second dosage. As an example, each subsequent second dosage can be given in approximate 8 hours to 14 hours intervals. In some embodiments, subsequent second dosages can be provided approximately every 12 hours after the initial second dosage. In some embodiments, each second dosage of compound (A), or a pharmaceutically acceptable salt thereof, can be given once daily. In other embodiments, each second dosage of compound (A), or a pharmaceutically acceptable salt thereof, can be given twice daily. One example of dosing is the first dosage of compound (A), or a pharmaceutically acceptable salt thereof, can be given once daily, and each second dosage of compound (A), or a pharmaceutically acceptable salt thereof, can be given twice daily.

In some embodiments, compound (A), or a pharmaceutically acceptable salt thereof, can be provided for a total number of at least 3 days. In other embodiments, compound (A), or a pharmaceutically acceptable salt thereof, can be provided for a total number of at least 5 days. In still other embodiments, compound (A), or a pharmaceutically acceptable salt thereof, can be provided for a total number of at least 7 days. In yet still other embodiments, compound (A), or a pharmaceutically acceptable salt thereof, can be provided for a total number of at least 14 days. In some embodiments, compound (A), or a pharmaceutically acceptable salt thereof, can be provided for a total number of at least 28 days. In some embodiments, compound (A), or a pharmaceutically acceptable salt thereof, can be provided for a total time period in the range of 3 days to 14 days. In other embodiments, compound (A), or a pharmaceutically acceptable salt thereof, can be provided for a total time period in the range of 3 days to 30 days. In some embodiments, the first dosage of compound (A), or a pharmaceutically acceptable salt thereof, can be given in a first time period (such as immediately after or within the first 12-24 hours following a positive diagnosis of a RSV infection) followed by several second dosages of compound (A), or a pharmaceutically acceptable salt thereof, for a second time period (for example, multiple days). In some embodiments, the second dosages of compound (A), or a pharmaceutically acceptable salt thereof, can be given for at least 3 days. In other embodiments, the second dosages of compound (A), or a pharmaceutically acceptable salt thereof, can be given for at least 4 days. In some embodiments, the second dosages of compound (A), or a pharmaceutically acceptable salt thereof, can be given for a number of days in the range of 3 to 7 days. In other embodiments, the second dosages of compound (A), or a pharmaceutically acceptable salt thereof, can be given for a number of days in the range of 3 to 14 days. In still other embodiments, the second dosages of compound (A), or a pharmaceutically acceptable salt thereof, can be given for a number of days in the range of 3 to 30 days.

Examples of regimens that include some of the embodiments described herein are provided in Tables 1, 2 and 3. The amounts in Tables 1 and 2 are for compound (A), or a pharmaceutically acceptable salt thereof, for use in adults. The amounts in Table 3 are for compound (A), or a pharmaceutically acceptable salt thereof, for use in children and infants.

TABLE 1

| Example | | No. of First | Each Second | Frequency of |
|---|---|---|---|---|
| Regimen | First Dosage | Dosages | Dosage | Second Dosages |
| 1 | 700 mg-800 mg | 1 | 100 mg-200 mg | 8-14 hour |
| | | | | intervals |
| 2 | 700 mg-800 mg | 1 | 450 mg-550 mg | 8-14 hour |
| | | | | intervals |
| 3 | 700 mg-800 mg | 1 | 325 mg-425 mg | 8-14 hour |
| | | | | intervals |
| 4 | 1400 mg-1600 mg | 2 | 100 mg-200 mg | 8-14 hour |
| | | each dosage | | intervals |
| | | 700 mg-800 mg | | |
| 5 | 1400 mg-1600 mg | 2 | 450 mg-550 mg | 8-14 hour |
| | | each dosage | | intervals |
| | | 700 mg-800 mg* | | |
| 6 | 1400 mg-1600 mg | 2 | 325 mg-425 mg | 8-14 hour |
| | | each dosage | | intervals |
| | | 700 mg-800 mg* | | |

*The two dosages are provided at different times (e.g., 1 dose in the morning and 1 dose at night).

TABLE 2

| Example | Dose Regimen | | | |
|---|---|---|---|---|
| Regimen | (mg) | Dose 1 | Dose 2 | Doses 3-10 |
| 7 | 100 (bid) | 100 mg (bid) | | |
| 8 | 100 (qd) | 100 mg (qd) | | |
| 9 | 325/80 | 325 mg | 80 mg | 80 mg (bid) |
| 10 | 400/100 | 400 mg | 100 mg | 100 mg (bid) |
| 11 | 200/200/100 | 200 mg | 200 mg | 100 mg (bid) |
| 12 | 750/750/500 | 750 mg | 750 mg | 500 mg (bid) |
| 13 | 750/500 | 750 mg | 500 mg | 500 mg (bid) |
| 14 | 200/50 | 200 mg | 50 mg | 50 mg (bid) |
| 15 | 400/50 | 400 mg | 50 mg | 50 mg (bid) |
| 16 | 400/0/50 | 400 mg | 0 mg | 50 mg (bid) |
| 17 | 50 (bid) | 50 mg (bid) | | |
| 18 | 200 (qd) | 200 mg (qd) | | |
| 19 | 400 (qd) | 400 mg (qd) | | |
| 20 | 500 (bid) | 500 mg (bid) | | |
| 21 | 100 q6h (bid) | 100 mg q6h (bid) | | 100 mg (bid) |
| 22 | 200 q6h (bid) | 200 mg q6h (bid) | | 200 mg (bid) |
| 23 | 400 q6h (bid) | 400 mg q6h (bid) | | 400 mg (bid) |
| 24 | 750/150 | 750 mg | 150 mg | 150 mg (bid) |

TABLE 3

| Example | Dose Regimen | | | |
|---|---|---|---|---|
| Regimen | (mg) | Dose 1 | Dose 2 | Doses 3-10 |
| 25 | 10 mg/kg (bid) | 10 mg/kg | | |
| 26 | 25 mg/kg (bid) | 25 mg/kg | | |
| 27 | 25/5 mg/kg | 25 mg/kg | 5 mg/kg | 5 mg/kg (bid) |
| 28 | 10/2 mg/kg | 10 mg/kg | 2 mg/kg | 2 mg/kg (bid) |
| 29 | 50/10 mg/kg | 50 mg/kg | 10 mg/kg | 10 mg/kg (bid) |
| 30 | 30/6 mg/kg | 30 mg/kg | 6 mg/kg | 6 mg/kg (bid) |
| 31 | 25/5 mg/kg (qd) | 25 mg/kg | | 5 mg/kg (qd) |
| 32 | 50/10 mg/kg | 50 mg/kg | | 10 mg/kg (qd) |
| | (qd) | | | |
| 33 | 25 mg/kg (qd) | 25 mg/kg | | 25 mg/kg (qd) |
| 34 | 10 mg/kg (qd) | 10 mg/kg | | 10 mg/kg (qd) |

Some embodiments described herein relate to a method for ameliorating or treating a paramyxovirus infection that can include administering a first dosage of compound (A), or a pharmaceutically acceptable salt thereof, and administering multiple separate second dosages of compound (A), or a pharmaceutically acceptable salt thereof, to a subject suffering from the paramyxovirus infection, wherein the first dosage and the multiple separate second dosages are provided according to a regimen selected from 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33 and 34 in Tables 1, 2, and/or 3. Other embodiments described herein relate to using a first dosage compound (A), or a pharmaceutically acceptable salt thereof, and multiple separate second dosages of compound (A), or a pharmaceutically acceptable salt thereof, in the manufacture of a medicament for ameliorating and/or treating a paramyxovirus infection in a subject suffering from the paramyxovirus infection, wherein the first dosage and the multiple separate second dosages are provided according to a regimen selected from 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33 and 34 in Tables 1, 2, and/or 3. Still other embodiments described herein relate to a first dosage of compound (A), or a pharmaceutically acceptable salt thereof, and multiple separate second dosages of compound (A), or a pharmaceutically acceptable salt thereof, that can be used for ameliorating and/or treating a paramyxovirus infection in a subject suffering from the paramyxovirus infection, wherein the first dosage and the multiple separate second dosages are provided according to a regimen selected from 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33 and 34 in Tables 1, 2, and/or 3.

Various routes are suitable for providing compound (A), or a pharmaceutically acceptable salt thereof. In some embodiments, compound (A), or a pharmaceutically acceptable salt thereof, can be provided in an oral dosage form. Any orally acceptable dosage form including, but not limited to, capsules, tablets, pills, powders, granules, emulsions, microemulsions, suspensions (e.g., aqueous suspensions), syrups, elixirs, or solutions can be used to provide compound (A), or a pharmaceutically acceptable salt thereof. Liquid dosage forms for oral administration include, but are not limited to, pharmaceutically acceptable emulsions, microemulsions, solutions, suspensions, syrups and elixirs. Solid dosage forms for oral administration include capsules (for example, soft and hard-filled gelatin capsules), tablets, pills, powders, and granules. The oral dosage forms can be prepared using methods known to those skilled in the art and may contain additional materials such as pharmaceutically acceptable excipient(s) or carrier(s).

Compound (A), or a pharmaceutically acceptable salt thereof, can be used in combination with one or more anti-RSV agents. One suitable anti-RSV agent is GS-5806 (N-(2-((S)-2-(5-((S)-3-Aminopyrrolidin-1-yl)-6-methylpyrazolo[1,5-a]pyrimidin-2-yl)piperidine-1-carbonyl)-4-chlorophenyl)methanesulfonamide), or a pharmaceutically acceptable salt thereof, (Gilead Sciences). GS-5806 is a RSV fusion inhibitor that can be given orally. As with compound (A), or a pharmaceutically acceptable salt thereof, GS-5806, or a pharmaceutically acceptable salt thereof, can be given at various dosages, frequency and length of time.

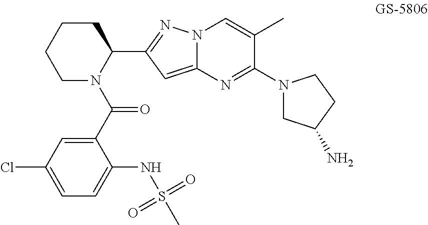 [SMILES: Cc1cn2nc([C@@H]3CCCCN3C(=O)c3cc(Cl)ccc3NS(C)(=O)=O)cc2nc1N1CC[C@H](N)C1]

Examples of suitable amounts of GS-5806, or a pharmaceutically acceptable salt thereof, include, but are not limited to, the following embodiments. In some embodiments, GS-5806, or a pharmaceutically acceptable salt thereof, can be administered to a subject suffering from RSV in an amount in the range of 75 mg to 100 mg, in combination with compound (A), or a pharmaceutically acceptable salt thereof. In other embodiments, GS-5806, or a pharmaceutically acceptable salt thereof, can be administered to a subject suffering from RSV in an amount in the range of 75 mg to 125 mg, in combination with compound (A), or a pharmaceutically acceptable salt thereof. In still other embodiments, GS-5806, or a pharmaceutically acceptable salt thereof, can be administered to a subject suffering from RSV in an amount in the range of 5 mg to 10 mg, in combination with compound (A), or a pharmaceutically acceptable salt thereof. In yet still other embodiments, GS-5806, or a pharmaceutically acceptable salt thereof, can be administered to a subject suffering from RSV in an amount in the range of 2.5 mg to 8 mg, in combination with compound (A), or a pharmaceutically acceptable salt thereof. In some embodiments, GS-5806, or a pharmaceutically acceptable salt thereof, can be administered to a subject suffering from RSV in an amount in the range of 10 mg to 75 mg, in combination with compound (A), or a pharmaceutically acceptable salt thereof. In other embodiments, GS-5806, or a pharmaceutically acceptable salt thereof, can be administered to a subject suffering from RSV in an amount in the range of 25 mg to 50 mg, in combination with compound (A), or a pharmaceutically acceptable salt thereof. In still other embodiments, GS-5806, or a pharmaceutically acceptable salt thereof, can be administered to a subject suffering from RSV in an amount in the range of 150 mg to 250 mg, in combination with compound (A), or a pharmaceutically acceptable salt thereof. In yet still other embodiments, GS-5806, or a pharmaceutically acceptable salt thereof, can be administered to a subject suffering from RSV in an amount in the range of 125 mg to 225 mg, in combination with compound (A), or a pharmaceutically acceptable salt thereof.

In some embodiments, GS-5806, or a pharmaceutically acceptable salt thereof, can be administered to a subject suffering from RSV in an amount in the range of 0.5 mg/kg to 10 mg/kg, in combination with compound (A), or a pharmaceutically acceptable salt thereof. In other embodiments, GS-5806, or a pharmaceutically acceptable salt thereof, can be administered to a subject suffering from RSV in an amount in the range of 1 mg/kg to 7 mg/kg, in combination with compound (A), or a pharmaceutically acceptable salt thereof. In still other embodiments, GS-5806, or a pharmaceutically acceptable salt thereof, can be administered to a subject suffering from RSV in an amount in the range of 1.5 mg/kg to 5 mg/kg, in combination with compound (A), or a pharmaceutically acceptable salt thereof.

As with compound (A), a first dosage of GS-5806, or a pharmaceutically acceptable salt thereof, can be administered, followed by several separate second dosages of GS-5806, or a pharmaceutically acceptable salt thereof. Suitable amounts of GS-5806, or a pharmaceutically acceptable salt thereof, for the first and second dosages are provided herein. In some embodiments, the first dosage of GS-5806, or a pharmaceutically acceptable salt thereof, can be provided in multiple dosages. The multiple dosages can be taken together at a first time period. Alternatively, at least one dosage form of the multiple dosages of the first dosage can be taken at a first time period and at least one dosage form of the multiple dosage forms of the first dosage can be taken at a second time period (for example, twice daily).

Examples of suitable regimens using GS-5806 that can be used in combination with any of the regimens described herewith with respect to compound (A), or a pharmaceutically acceptable salt thereof, include those provided in Table 4. The amounts in Table 4 are for GS-5806, or a pharmaceutically acceptable salt thereof.

TABLE 4

| | | | | Period |
|---|---|---|---|---|
| | | | | of Time |
| Example | First Dosage | Period of Time | Each Second | for Second |
| Regimen | (Day 1) | for First Dosage | Dosage | Dosages |
| 1 | 50 mg | 1 day | 25 mg (daily) | 4 days |
| 2 | 100 mg | 1 day | 5 mg (daily) | 4 days |
| 3 | 10 mg | 1 day | 5 mg (daily) | 4 days |
| 4 | 50 mg | 1 day | 25 mg (daily) | 2 days |
| 5 | 200 mg^{a} | 1 day | | |

^{a}can be given in a single dosage form or multiple dosage forms (e.g., 4 × 50 mg)

The order of administration of the compounds in a combination therapy (for example, a compound (A) and GS-5806, or a pharmaceutically acceptable salt of the foregoing) can vary. In some embodiments, compound (A), or a pharmaceutically acceptable salt thereof, can be administered prior to all compounds of the combination therapy. In other embodiments, compound (A), or a pharmaceutically acceptable salt thereof, can be administered prior to at least one compound of the combination therapy. In still other embodiments, compound (A), or a pharmaceutically acceptable salt thereof, can be administered concomitantly with one or more compound(s) of the combination therapy. In yet still other embodiments, compound (A), or a pharmaceutically acceptable salt thereof, can be administered subsequent to the administration of at least one compound of the combination therapy. In some embodiments, compound (A), or a pharmaceutically acceptable salt thereof, can be administered subsequent to the administration of all other compounds of the combination therapy.

In some embodiments, a combination of compound (A) and GS-5806, or a pharmaceutically acceptable salt of the foregoing, can result in an additive effect. In some embodiments, a combination of compound (A) and GS-5806, or a pharmaceutically acceptable salt of the foregoing, can result in a synergistic effect. In some embodiments, a combination of compound (A) and GS-5806, or a pharmaceutically acceptable salt of the foregoing, can result in a strongly synergistic effect. In some embodiments, a combination of compound (A) and GS-5806, or a pharmaceutically acceptable salt of the foregoing, is not antagonistic.

As used herein, the term “antagonistic” means that the activity of the combination of compounds is less compared to the sum of the activities of the compounds in combination when the activity of each compound is determined individually (i.e., as a single compound). As used herein, the term “synergistic effect” means that the activity of the combination of compounds is greater than the sum of the individual activities of the compounds in the combination when the activity of each compound is determined individually. As used herein, the term “additive effect” means that the activity of the combination of compounds is about equal to the sum of the individual activities of the compounds in the combination when the activity of each compound is determined individually.

A potential advantage of utilizing a combination of compound (A) and GS-5806, or a pharmaceutically acceptable salt of the foregoing, may be a reduction in the required amount(s) of the compound(s) that is effective in treating RSV, as compared to the amount required to achieve same therapeutic result when the compound(s), is administered as monotherapy. For example, the amount of compound (A) and/or GS-5806, or a pharmaceutically acceptable salt of the foregoing, in a combination described herein can be less compared to the amount of compound (A) and/or GS-5806, or a pharmaceutically acceptable salt of the foregoing, needed to achieve the same viral load reduction when administered as a monotherapy. Another potential advantage of utilizing a combination of compound (A) and GS-5806, or a pharmaceutically acceptable salt of the foregoing, is that the use of two or more compounds having different mechanisms of action can create a higher barrier to the development of resistant viral strains compared to the barrier when a compound is administered as monotherapy. Additional advantages of utilizing a combination of compound (A) and GS-5806, or a pharmaceutically acceptable salt of the foregoing, may include little to no cross resistance between the compounds of the combination; different routes for elimination; little to no overlapping toxicities; little to no significant effects on cytochrome P450; and/or little to no pharmacokinetic interactions between the compounds of the combination.

As used herein, the terms “treat,” “treating,” “treatment,” “therapeutic,” and “therapy” do not necessarily mean total cure or abolition of the disease or condition. Any alleviation of any undesired signs or symptoms of a disease or condition, to any extent can be considered treatment and/or therapy. Furthermore, treatment may include acts that may worsen the subject's overall feeling of well-being or appearance.

As used herein, the terms “prevent” and “preventing,” mean lowering the efficiency of viral replication and/or inhibiting viral replication to a greater degree in a subject who receives the compound compared to a subject who does not receive the compound. Examples of forms of prevention include prophylactic administration to a subject who has been or may be exposed to an infectious agent, such as a paramyxovirus (e.g., RSV).

As used herein, a “subject” refers to an animal that is the object of treatment, observation or experiment. “Animal” includes cold- and warm-blooded vertebrates and invertebrates such as fish, shellfish, reptiles and, in particular, mammals. “Mammal” includes, without limitation, mice, rats, rabbits, guinea pigs, dogs, cats, sheep, goats, cows, horses, primates, such as monkeys, chimpanzees, and apes, and, in particular, humans. In some embodiments, the subject can be an adult human (18 years or older). In other embodiments, the subject can be child (>1-17 years). In still other embodiments, the subject can be an infant (1 year and younger).

The terms “therapeutically effective amount” and “effective amount” are used to indicate an amount of an active compound, or pharmaceutical agent, that elicits the biological or medicinal response indicated. For example, an effective amount of compound can be the amount needed to prevent, alleviate or ameliorate symptoms of disease or prolong the survival of the subject being treated This response may occur in a tissue, system, animal or human and includes alleviation of the signs or symptoms of the disease being treated. Determination of an effective amount is well within the capability of those skilled in the art, in view of the disclosure provided herein. The therapeutically effective amount of the compounds disclosed herein required as a dose will depend on the route of administration, the type of animal, including human, being treated, and the physical characteristics of the specific animal under consideration. The dose can be tailored to achieve a desired effect, but will depend on such factors as weight, diet, concurrent medication and other factors which those skilled in the medical arts will recognize.

Various indicators for determining the effectiveness of a method for treating a RSV viral infection are known to those skilled in the art. Example of suitable indicators include, but are not limited to, a reduction in viral load, a reduction in viral replication, a reduction in time to seroconversion (virus undetectable in patient serum), a reduction of morbidity or mortality in clinical outcomes, and/or other indicator of disease response.

In some embodiments, an effective amount of compound (A) and/or compound (B), or a pharmaceutically acceptable salt of the foregoing, is an amount that is effective to reduce viral titers to undetectable levels, for example, less than 1.7 log₁₀ plaque forming units equivalents (PFUe)/mL, or less than 0.3 log₁₀ plaque forming units equivalents (PFUe)/mL. In some embodiments, an effective amount of compound (A) and/or compound (B), or a pharmaceutically acceptable salt of the foregoing, is an amount that is effective to reduce viral load compared to the viral load before administration of compound (A), or a pharmaceutically acceptable salt thereof. For example, the viral load is measure before administration of compound (A), or a pharmaceutically acceptable salt thereof, and again several hours after receiving the initial dosage of compound (A), or a pharmaceutically acceptable salt thereof (for example, 60 hours after receiving the initial dosage of compound (A), or a pharmaceutically acceptable salt thereof). In some embodiments, compound (A) and/or compound (B), or a pharmaceutically acceptable salt of the foregoing, can be an amount that is effective to reduce viral load to lower than 1.7 log₁₀ (PFUe)/mL, or lower than 0.3 log₁₀ (PFUe)/mL. In some embodiments, an effective amount of compound (A) and/or compound (B), or a pharmaceutically acceptable salt of the foregoing, is an amount that is effective to achieve a reduction in viral titer in the serum of the subject in the range of about 1.5-log to about a 2.5-log reduction, about a 3-log to about a 4-log reduction, or a greater than about 5-log reduction compared to the viral load before administration of compound (A), or a pharmaceutically acceptable salt thereof. For example, the viral load is measure before administration of compound (A), or a pharmaceutically acceptable salt thereof, and several hours after receiving the initial dosage of compound (A), or a pharmaceutically acceptable salt thereof (for example, 60 hours after receiving the initial dosage of compound (A), or a pharmaceutically acceptable salt thereof).

In some embodiments, compound (A) and/or compound (B), or a pharmaceutically acceptable salt of the foregoing, can result in at least a 1, 2, 3, 4, 5, 10, 15, 20, 25, 50, 75, 100-fold or more reduction in the replication of RSV relative to pre-treatment levels in a subject, as determined several hours after receiving the initial dosage of compound (A), or a pharmaceutically acceptable salt thereof (for example, 60 hours after receiving the initial dosage of compound (A), or a pharmaceutically acceptable salt thereof). In some embodiments, compound (A) and/or compound (B), or a pharmaceutically acceptable salt of the foregoing, can result in a reduction of the replication of RSV relative to pre-treatment levels in the range of about 2 to about 5 fold, about 10 to about 20 fold, about 15 to about 40 fold, or about 50 to about 100 fold. In some embodiments, compound (A) and/or compound (B), or a pharmaceutically acceptable salt of the foregoing, can result in a reduction of RSV replication in the range of 1 to 1.5 log, 1.5 log to 2 log, 2 log to 2.5 log, 2.5 to 3 log, 3 log to 3.5 log or 3.5 to 4 log more reduction of RSV replication compared to the reduction of RSV reduction achieved by ribavirin (Virazole®), or may achieve the same reduction as that of ribavirin (Virazole®) therapy in a shorter period of time, for example, in one day, two days, three days, four days, or five days, as compared to the reduction achieved after 5 days of ribavirin (Virazole®) therapy.

In some embodiments, an effective amount of compound (A) and/or compound (B), or a pharmaceutically acceptable salt of the foregoing, is an amount that is effective to achieve an undetectable level of viral RNA in less than 5 days (120 hours) after the initial administration of the first dosage. In some embodiments, an effective amount of compound (A) and/or compound (B), or a pharmaceutically acceptable salt of the foregoing, is an amount that is effective to achieve an undetectable level of viral RNA in less than 3 days (72 hours) after the initial administration of the first dosage.

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. In the event that there are a plurality of definitions for a term herein, those in this section prevail unless stated otherwise.

The term “pharmaceutically acceptable salt” refers to a salt of a compound that does not cause significant irritation to an organism to which it is administered and does not abrogate the biological activity and properties of the compound. In some embodiments, the salt is an acid addition salt of the compound. Pharmaceutical salts can be obtained by reacting a compound with inorganic acids such as hydrohalic acid (e.g., hydrochloric acid or hydrobromic acid), sulfuric acid, nitric acid and phosphoric acid. Pharmaceutical salts can also be obtained by reacting a compound with an organic acid such as aliphatic or aromatic carboxylic or sulfonic acids, for example formic, acetic, succinic, lactic, malic, tartaric, citric, ascorbic, nicotinic, methanesulfonic, ethanesulfonic, p-toluensulfonic, salicylic or naphthalenesulfonic acid. Pharmaceutical salts can also be obtained by reacting a compound with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, C₁-C₇ alkylamine, cyclohexylamine, triethanolamine, ethylenediamine, and salts with amino acids such as arginine and lysine.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; and use of terms like ‘preferably,’ ‘preferred,’ ‘desired,’ or ‘desirable,’ and words of similar meaning should not be understood as implying that certain features are critical, essential, or even important to the structure or function, but instead as merely intended to highlight alternative or additional features that may or may not be utilized in a particular embodiment. In addition, the term “comprising” is to be interpreted synonymously with the phrases “having at least” or “including at least”. When used in the context of a process, the term “comprising” means that the process includes at least the recited steps, but may include additional steps. When used in the context of a compound, composition or device, the term “comprising” means that the compound, composition or device includes at least the recited features or components, but may also include additional features or components. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. Any reference signs in the claims should not be construed as limiting the scope.

It is understood that, in any compound described herein having one or more chiral centers, if an absolute stereochemistry is not expressly indicated, then each center may independently be of R-configuration or S-configuration or a mixture thereof. Thus, the compounds provided herein may be enantiomerically pure, enantiomerically enriched, racemic mixture, diastereomerically pure, diastereomerically enriched, or a stereoisomeric mixture. In addition it is understood that, in any compound described herein having one or more double bond(s) generating geometrical isomers that can be defined as E or Z, each double bond may independently be E or Z a mixture thereof.

Likewise, it is understood that, in any compound described, all tautomeric forms are also intended to be included. Additionally, all tautomers of heterocyclic bases known in the art are intended to be included, including tautomers of natural and non-natural purine-bases and pyrimidine-bases.

It is to be understood that where compounds disclosed herein have unfilled valencies, then the valencies are to be filled with hydrogens or isotopes thereof, e.g., hydrogen-1 (protium) and hydrogen-2 (deuterium).

It is understood that the compounds described herein can be labeled isotopically. Substitution with isotopes such as deuterium may afford certain therapeutic advantages resulting from greater metabolic stability, such as, for example, increased in vivo half-life or reduced dosage requirements. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium) and hydrogen-2 (deuterium). Thus, reference herein to a compound encompasses all potential isotopic forms unless the context clearly dictates otherwise.

It is understood that the methods and combinations described herein include crystalline forms (also known as polymorphs, which include the different crystal packing arrangements of the same elemental composition of a compound), amorphous phases, salts, solvates, and hydrates. In some embodiments, the compounds described herein exist in solvated forms with pharmaceutically acceptable solvents such as water, ethanol, or the like. In other embodiments, the compounds described herein exist in unsolvated form. Solvates contain either stoichiometric or non-stoichiometric amounts of a solvent, and may be formed during the process of crystallization with pharmaceutically acceptable solvents such as water, ethanol, or the like. Hydrates are formed when the solvent is water, or alcoholates are formed when the solvent is alcohol. In addition, the compounds provided herein can exist in unsolvated as well as solvated forms. In general, the solvated forms are considered equivalent to the unsolvated forms for the purposes of the compounds and methods provided herein.

Where a range of values is provided, it is understood that the upper and lower limit, and each intervening value between the upper and lower limit of the range is encompassed within the embodiments.

### EXAMPLES

### Example 1

### Treatment Regimens

Healthy adults received one of the following dosing regimens or placebo over 5 days using the human RSV challenge model.

| Regimen | First Dosage | Second Dosages | |
|---|---|---|---|
| 1 | 750 mg (single dosage) | 150 mg | N = 7 |
| 2 | 750 mg (single dosage) | 500 mg | N = 8 |

| | | |
|---|---|---|
| 3 | 375 mg | N = 8 |
| 4 | Placebo | N = 12 |

Subjects were given an intranasal inoculation of RSV-A Memphis 37b challenge virus. Administration of compound (A), or a pharmaceutically acceptable salt thereof, began approximately 12 hours after confirmation of RSV infection as determined by the presence of RSV RNA in nasopharyngeal washes. The test compound was administered as an oral-liquid suspension, wherein the drug vehicle was methyl cellulose and sterile water. The placebo was the drug vehicle without the test compound. Second dosages were started 12 hours after administration of the first dosage, and the remaining second dosages were provided in approximate 12 hour intervals. Nasal washes were collected twice daily approximately 36 to 48 hours after RSV inoculation until Day 12. Viral load was detected and quantified from the aliquots of the nasal wash samples using tissue infectivity plaque assays and PCR. (See DeVincenzo, J. P., et al., Am. J. Respir. Crit. Care. Med. (2010) 182(10):1305-1314) Subjects returned for two follow-up visits on Day 16 (±2 days) and Day 28 (±2 days) post-challenge inoculation.

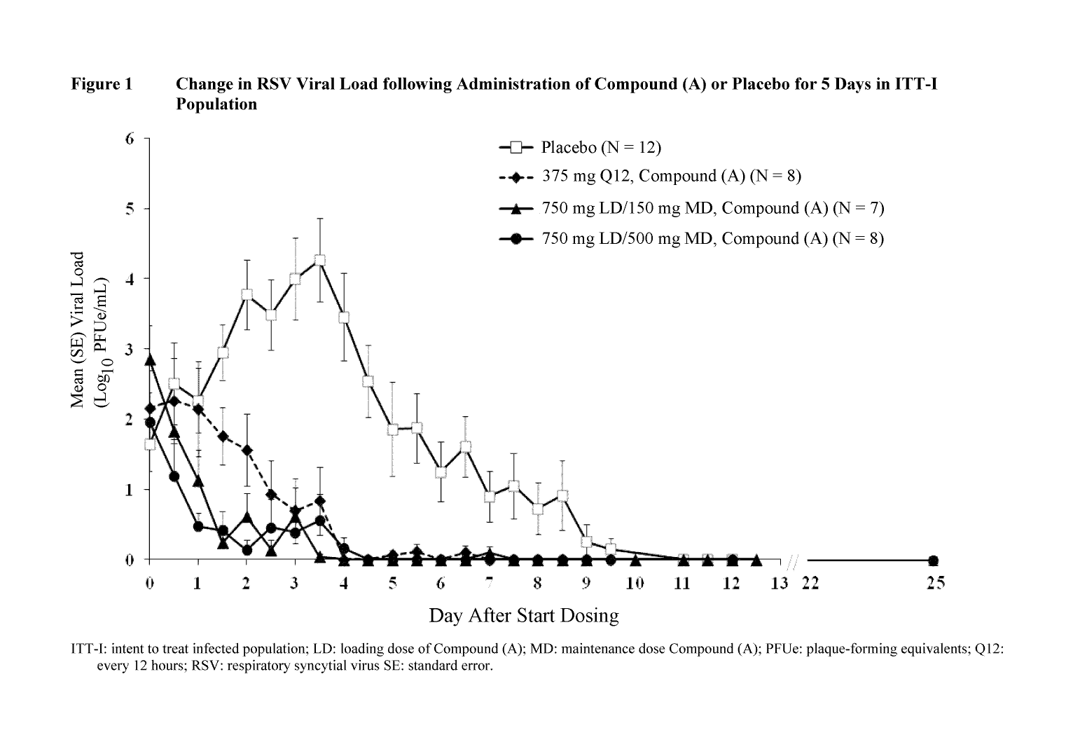

As shown in FIG. 1, all regimens with Compound (A), or a pharmaceutically acceptable salt thereof, resulted in the marked reduction in RSV viral load compared to the placebo. The placebo group exhibited a logarithmic increase in RSV RNA with a peak viral load at approximately Day 3.5 following the start of dosing. At Day 12, all subject treated with compound (A), or a pharmaceutically acceptable salt thereof, had undetectable RSV RNA. In contrast, the subjects receiving the placebo had a mean RSV RNA of 0.52 log₁₀ plaque forming units equivalents (PFUe)/mL on Day 12. At both Day 16 and 28, RSV RNA remained undetectable in the subjects who received compound (A), or a pharmaceutically acceptable salt thereof. Those subjects who received 750 mg of compound (A), or a pharmaceutically salt thereof, on Day 1 had a multi-log reduction of plaque forming units equivalents (PFUe/mL) within the first 24 hours. Additionally, no subject discontinued treatment during the study and no clinically significant laboratory abnormalities were observed. Thus, compound (A), or a pharmaceutically acceptable salt thereof, provides a significant advancement for treating RSV.

### Example 2

### Treatment Regimens

Within the clinical development program, a range of doses and durations are evaluated in adults with a RSV infection using a compound described herein (for example, compound (A), or a pharmaceutically acceptable salt thereof). For example, subjects receive one of the following orally administered dosing regimens over 5-7 days in a randomized clinical trial.

| Dose Regimen | | | |
|---|---|---|---|
| (mg) | Dose 1 | Dose 2 | Doses 3-10 |
| 750/500 mg | 750 mg | 500 mg | 500 mg (bid) |
| 750/150 mg | 750 mg | 150 mg | 150 mg (bid) |
| 500/150 mg | 500 mg | 150 mg | 150 mg (bid) |

### Example 3

### Treatment Regimens

Within the clinical development program, a range of doses and durations are evaluated in infants and children with a RSV infection using a compound described herein (for example, compound (A), or a pharmaceutically acceptable salt thereof). For example, subjects receive one of the following orally administered dosing regimens over 5-7 days in a randomized clinical trial.

| Dose Regimen | | | |
|---|---|---|---|
| (mg/kg) | Dose 1 | Dose 2 | Doses 3-10 |
| 25/5 mg/kg | 25 mg/kg | 5 mg/kg | 5 mg/kg (bid) |
| 10/2 mg/kg | 10 mg/kg | 2 mg/kg | 2 mg/kg (bid) |
| 50/10 mg/kg | 50 mg/kg | 10 mg/kg | 10 mg/kg (bid) |

### Example 4

### Combination Studies

RSV with Renilla Reporter

RSV expressing Renilla luciferase (A2-RL-line19F) are generated by Dr. Martin Moore of Emory University, Atlanta, Ga., USA. The in vitro viral kinetics of A2-RL-line19F is similar to that of wild type RSV (See Hotard, A. L., Virology (2012) 434(1):129-136).

Host cell HEp-2 is purchased from ATCC (Cat. #CCL-23) and cells are cultured in DMEM/Ham's F-12 50/50 1× containing L-glutamine and 15 mM HEPES (Mediatech, Cat. #10-092-CM). The medium is further supplemented with 5% (v/v) FBS (Mediatech, Cat. #35-010-CV) and 1% (v/v) penicillin/streptomycin (Mediatech, Cat. #30-002-0). HEp-2 cells are maintained at 37° C. in a humidified 5% CO₂ atmosphere.

### Drug Treatment and Viral Dosing

To determine the effect of a combination of compounds, the following procedure is followed. On the first day, 20,000 HEp-2 cells are plated per well in a 96-well plate. On the following day, test articles are solubilized in 100% DMSO (for chemicals) or 1×PBS (for biologics) to 200× the desired final testing concentration. Subsequently, Compound (A), or a pharmaceutically acceptable salt thereof, is serially diluted (1:3) to 9 distinct concentrations “horizontally” in a 96-well plate, and the second test compound is serially diluted (1:3) to 7 distinct concentrations “vertically” in 96-well plate. The serially diluted 200× test articles are then diluted 1:10 into cell culture media to generate 20× test articles. A 5 μL aliquot of the 20× test articles is added in a checkerboard fashion to the cells with 90 μL existing media. Space is also allotted for titrations of each of the compounds alone to be used as reference controls. After 12 hour pre-incubation of test articles, A2-RL-line19F at an MOI of 0.5 is added to the plate and further incubated for 2 days at 37° C. in a 5% CO₂.

### Determination of Anti-RSV Activity

The Renilla Luciferase Assay System (Promega, Cat. # E2820) is used to measure anti-RSV replicon activity. Assay plates were set up as stated above. Luminescence is recorded using a Perkin Elmer multilabel counter Victor3V.

### Cell Viability Assay

Promega CellTiter-Glo Luminescent Cell Viability Assay, Cat. #G7572) is used to measure cell viability. The CellTiter-Glo® Luminescent Cell Viability Assay is a homogeneous method to determine the number of viable cells in culture based on quantitation of the adenosine triphosphate (ATP) present, which signals the presence of metabolically active cells. Assay plates are set up in the same format the anti-RSV assay, except that no virus is added to the cell viability assay. A 100-μL, aliquot of CellTiter-Glo reagent is added to each well and incubated at room temperature for 8 minutes. Luminescence is recorded using a Perkin Elmer multilabel counter Victor3V.

### Data Analysis

Each experiment is performed at N=5 for both anti-RSV activity and cell viability. Mean percent inhibition of the replicon values from the 5 experiments is generated and for anti-RSV activity, it is analyzed using two drug interaction analysis models, Isobologram Analysis and/or Prichard's Model.

### Isobologram Analysis

The effects of drug-drug combinations are evaluated by the Loewe additivity model in which the experimental data are analyzed using CalcuSyn (Biosoft, Ferguson, Mo.), a computer program based on the method of Chou and Talalay. The combination index (CI) value and the isobologram for each experimental combination are calculated. CI values of <1, 1, and >1 indicate synergy, additive effect, and antagonism, respectively. Under the synergy category, CI<0.1 is considered very strong synergism; CI 0.1-0.3 strong synergism; CI 0.3-0.7 synergism and CI 0.7-0.85 moderate synergism. The isobologram analysis, which graphically represents additive, synergistic, and antagonistic drug effects, is also used to model the interaction of antiviral activities. In this representation, an effective concentration (EC) value of one drug is plotted on one axis and corresponding EC value of a second drug is plotted on the second axis; the line connecting these two points represents the amount of each drug in a combination that would be required to reach the equivalent EC value, given that their effects are additive.

Prichard's Model (MacSynergy II).

MacSynergy II software is kindly provided by Dr. M. Prichard (University of Michigan). This program allows the three-dimensional examination of drug interactions of all data points generated from the checkerboard combination of two inhibitors with Bliss-Independence model. Confidence bounds are determined from replicate data. If the 95% confidence limits (CL) do not overlap the theoretic additive surface, then the interaction between the two drugs differs significantly from additive. The volumes of synergy or antagonism can be determined and graphically depicted in three dimensions and represent the relative quantity of synergism or antagonism per change in the two drug concentrations. Synergy and antagonism volumes are based on the Bliss independence model, which assumes that both compounds act independently on different targets. A set of predicted fractional responses faAB under the Bliss independence model is calculated as faAB=faA+faB−fail·faB with faA and faB representing the fraction of possible responses, e.g. % inhibition, of compounds A and B at amounts dA and dB, respectively, and describes the % inhibition of a combination of compounds A and B at amount (dA+dB). If faAB>faA+faB−faA·faB then we have Bliss synergy; if faAB<faA+faB−faA·faB then we have Bliss antagonism. The 95% synergy/antagonism volumes are the summation of the differences between the observed inhibition and the 95% confidence limit on the prediction of faAB under the Bliss independence model. Table 1 shows the volumes and corresponding volume descriptions for the results of the Bliss Independence Analysis. MacSynergy II is used for data analysis.

TABLE 1
MacSynergy II Volume Descriptions

| Volume (μM² %) | Volume Description |
|---|---|
| <25 | Additive |
| 25-50 | Minor synergism |
| 50-100 | Significant synergism |
| >100 | Strong synergism |

Furthermore, although the foregoing has been described in some detail by way of illustrations and examples for purposes of clarity and understanding, it will be understood by those of skill in the art that numerous and various modifications can be made without departing from the spirit of the present disclosure. Therefore, it should be clearly understood that the forms disclosed herein are illustrative only and are not intended to limit the scope of the present disclosure, but rather to also cover all modification and alternatives coming with the true scope and spirit of the invention.

1. A method for ameliorating or treating a paramyxovirus infection comprising administering compound (A), or a pharmaceutically acceptable salt thereof, in a first dosage and administering compound (A), or a pharmaceutically acceptable salt thereof, in multiple separate second dosages; and wherein compound (A) is

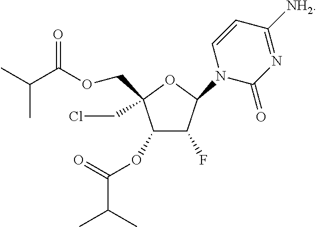 [SMILES: CC(C)C(=O)OC[C@@]1(CCl)O[C@@H](n2ccc(N)nc2=O)[C@H](F)[C@@H]1OC(=O)C(C)C]

2. The method of claim 1, wherein compound (A), or a pharmaceutically acceptable salt thereof, provides compound (B) having the structure

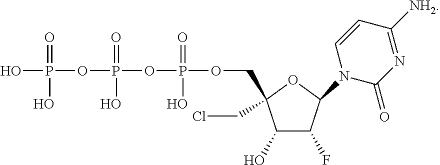 [SMILES: Nc1ccn([C@@H]2O[C@](CCl)(COP(=O)(O)OP(=O)(O)OP(=O)(O)O)[C@@H](O)[C@H]2F)c(=O)n1]

3. The method of claim 1, wherein the first dosage of compound (A), or a pharmaceutically acceptable salt thereof, is a loading dosage and each separate second dosage of compound (A), or a pharmaceutically acceptable salt thereof, is a maintenance dosage.

4. The method of claim 1, wherein the first dosage of compound (A), or a pharmaceutically acceptable salt thereof, includes an amount of compound (A), or a pharmaceutically acceptable salt thereof, in the range of 700 mg to 1600 mg.

5. The method of claim 1, wherein the first dosage of compound (A), or a pharmaceutically acceptable salt thereof, includes an amount of compound (A), or a pharmaceutically acceptable salt thereof, in the range of 5 mg/kg to 75 mg/kg.

6. (canceled)

7. The method of claim 1, wherein the first dosage is provided in multiple dosages.

8. The method claim 7, wherein the first dosage of compound (A), or a pharmaceutically acceptable salt thereof, is provided in two dosages, and wherein each of the two dosages includes an amount of compound (A), or a pharmaceutically acceptable salt thereof, in the range of 700 mg to 800 mg.

9. (canceled)

10. The method of claim 7, wherein two dosages are given twice daily.

11. The method of claim 1, wherein the first dosage is provided in a single dosage.

12. The method of claim 11, wherein the single dosage includes an amount of compound (A), or a pharmaceutically acceptable salt thereof, in the range of 700 mg to 800 mg.

13. (canceled)

14. The method of claim 11, wherein the single dosage includes an amount of compound (A), or a pharmaceutically acceptable salt thereof, in the range of 450 mg to 550 mg.

15. (canceled)

16. The method of claim 11, wherein the single dosage includes an amount of compound (A), or a pharmaceutically acceptable salt thereof, in an amount in the range of 5 mg/kg to 75 mg/kg.

17. (canceled)

18. (canceled)

19. (canceled)

20. (canceled)

21. The method of claim 1, wherein each second dosage includes an amount of compound (A), or a pharmaceutically acceptable salt thereof, in an amount in the range of 450 mg to 550 mg.

22. (canceled)

23. The method of claim 1, wherein each second dosage includes an amount of compound (A), or a pharmaceutically acceptable salt thereof, in an amount in the range of 100 mg to 200 mg.

24. (canceled)

25. (canceled)

26. (canceled)

27. The method of claim 1, wherein each second dosage includes an amount of compound (A), or a pharmaceutically acceptable salt thereof, in an amount in the range of 1 mg/kg to 50 mg/kg.

28. (canceled)

29. (canceled)

30. (canceled)

31. (canceled)

32. (canceled)

33. The method of claim 1, wherein each second dosage is given twice daily.

34. The method of claim 1, wherein the initial second dosage is given about 12 hours after completion of the first dosage.

35. The method of claim 1, wherein the second dosages are given for a total of 3 days to 7 days after the completion of the first dosage.

36. (canceled)

37. The method of claim 1, wherein compound (A), or a pharmaceutically acceptable salt, is provided for a total number of days in the range of 3 days to 30 days.

38. (canceled)

39. The method of claim 1, wherein compound (A), or a pharmaceutically acceptable salt, is provided in an oral dosage form.

40. The method of claim 1, wherein compound (A), or a pharmaceutically acceptable salt, achieves an undetectable level of viral RNA in less than 5 days after the initial administration of the first dosage.

41. (canceled)

42. The method of claim 1, further comprising administering GS-5806, or a pharmaceutically salt thereof.

43. The method of claim 42, wherein N-(2-((S)-2-(5-((S)-3-Aminopyrrolidin-1-yl)-6-methylpyrazolo[1,5-a]pyrimidin-2-yl)piperidine-1-carbonyl)-4-chlorophenyl)methanesulfonamide, or a pharmaceutically salt thereof, is administered in an amount in the range of in the range of 0.5 mg/kg to 10 mg/kg.

44. The method of claim 42, wherein N-(2-((S)-2-(5-((S)-3-Aminopyrrolidin-1-yl)-6-methylpyrazolo[1,5-a]pyrimidin-2-yl)piperidine-1-carbonyl)-4-chlorophenyl)methane sulfonamide, or a pharmaceutically salt thereof, is administered in an amount of 200±10 mg.

45. (canceled)

46. A method for ameliorating or treating a paramyxovirus infection comprising administering compound (A), or a pharmaceutically acceptable salt thereof, in a first dosage and administering compound (A), or a pharmaceutically acceptable salt thereof, in multiple separate second dosages; and wherein compound (A) is 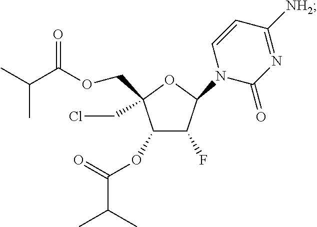 [SMILES: CC(C)C(=O)OC[C@@]1(CCl)O[C@@H](n2ccc(N)nc2=O)[C@H](F)[C@@H]1OC(=O)C(C)C]
and wherein the first dosage and the multiple separate second dosages are provided according to a regimen selected from 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33;

| | | No. of First | Each Second | Frequency of |
|---|---|---|---|---|
| Regimen | First Dosage | Dosages | Dosage | Second Dosages |
| 1 | 700 mg-800 mg | 1 | 100 mg-200 mg | 8-14 hour |
| | | | | intervals |
| 2 | 700 mg-800 mg | 1 | 450 mg-550 mg | 8-14 hour |
| | | | | intervals |
| 3 | 700 mg-800 mg | 1 | 325 mg-425 mg | 8-14 hour |
| | | | | intervals |
| 4 | 1400 mg-1600 mg | 2 | 100 mg-200 mg | 8-14 hour |
| | | each dosage | | intervals |
| | | 700 mg-800 mg | | |
| 5 | 1400 mg-1600 mg | 2 | 450 mg-550 mg | 8-14 hour |
| | | each dosage | | intervals |
| | | 700 mg-800 mg* | | |
| 6 | 1400 mg-1600 mg | 2 | 325 mg-425 mg | 8-14 hour |
| | | each dosage | | intervals |
| | | 700 mg-800 mg* | | |
| | Dose Regimen | | | |
|---|---|---|---|---|
| Regimen | (mg) | Dose 1 | Dose 2 | Doses 3-10 |
| 7 | 100 (bid) | 100 mg (bid) | | |
| 8 | 100 (qd) | 100 mg (qd) | | |
| 9 | 325/80 | 325 mg | 80 mg | 80 mg (bid) |
| 10 | 400/100 | 400 mg | 100 mg | 100 mg (bid) |
| 11 | 200/200/100 | 200 mg | 200 mg | 100 mg (bid) |
| 12 | 750/750/500 | 750 mg | 750 mg | 500 mg (bid) |
| 13 | 750/500 | 750 mg | 500 mg | 500 mg (bid) |
| 14 | 200/50 | 200 mg | 50 mg | 50 mg (bid) |
| 15 | 400/50 | 400 mg | 50 mg | 50 mg (bid) |
| 16 | 400/0/50 | 400 mg | 0 mg | 50 mg (bid) |
| 17 | 50 (bid) | 50 mg (bid) | | |
| 18 | 200 (qd) | 200 mg (qd) | | |
| 19 | 400 (qd) | 400 mg (qd) | | |
| 20 | 500 (bid) | 500 mg (bid) | | |
| 21 | 100 q6h (bid) | 100 mg q6h (bid) | | 100 mg (bid) |
| 22 | 200 q6h (bid) | 200 mg q6h (bid) | | 200 mg (bid) |
| 23 | 400 q6h (bid) | 400 mg q6h (bid) | | 400 mg (bid) |
| 24 | 750/150 | 750 mg | 150 mg | 150 mg (bid) |
| 25 | 10 mg/kg (bid) | 10 mg/kg | | |
| 26 | 25 mg/kg (bid) | 25 mg/kg | | |
| 27 | 25/5 mg/kg | 25 mg/kg | 5 mg/kg | 5 mg/kg (bid) |
| 28 | 10/2 mg/kg | 10 mg/kg | 2 mg/kg | 2 mg/kg (bid) |
| 29 | 50/10 mg/kg | 50 mg/kg | 10 mg/kg | 10 mg/kg (bid) |
| 30 | 30/6 mg/kg | 30 mg/kg | 6 mg/kg | 6 mg/kg (bid) |
| 31 | 25/5 mg/kg (qd) | 25 mg/kg | | 5 mg/kg (qd) |
| 32 | 50/10 mg/kg (qd) | 50 mg/kg | | 10 mg/kg (qd) |
| 33 | 25 mg/kg (qd) | 25 mg/kg | | 25 mg/kg (qd) |
| 34 | 10 mg/kg (qd) | 10 mg/kg | | 10 mg/kg(qd) |
| 25 | 10 mg/kg (bid) | 25 mg/kg | 5 mg/kg | 5 mg/kg (bid) |

47. The method of claim 1, wherein the paramyxovirus is RSV.

48. The method of claim 1, wherein the paramyxovirus is a human metapneumovirus.

49. The method of claim 1, wherein the paramyxovirus is a human parainfluenza virus.

50. The method of claim 49, wherein the human parainfluenza virus is HPIV-3.

51. The method of claim 1, wherein the subject is a human adult.

52. The method of claim 1, wherein the subject is a human child.

53. The method of claim 1, wherein the subject is a human infant.
